(19) United States
(12) Patent Application Publication
(10) Pub. No.: US 2008/0045949 A1
(43) Pub. Date: Feb. 21, 2008

(54) Method of treating degenerative spinal disorders

(76) Inventors: Margaret M. Hunt, Cedar Park, TX (US); David M. Hooper, Austin, TX (US); Alain Meunier, Bordeaux (FR); James Jara-Almonte, Kenosha, WI (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/455,401

(22) Filed: Jun. 19, 2006

Related U.S. Application Data
(60) Provisional application No. 60/691,711, filed on Jun. 17, 2005.

(51) Int. Cl. A61B 17/58

(52) U.S. Cl. 606/61; 606/76; 606/86

(57) ABSTRACT

The present invention describes methods for treating a degenerative intervertebral disc disorder comprising implanting a disc stabilization device into a subject and administering at least one therapeutic agent which promotes healing of the disc to the subject. The invention also includes methods of promoting healing of damaged or degenerated intervertebral discs comprising decreasing the load of the disc through a disc stabilization device and inhibiting the inflammatory process. Also described are hydrogels for use in combination with extradiscal stabilization devices, as intradiscal stabilization devices, as drug carriers, and as combinations thereof.

This application claims priority from U.S. Provisional Patent Application Ser. No. 60/691,711, filed Jun. 17, 2005, incorporated herein by reference.

### BACKGROUND OF THE INVENTION

The primary function of the intervertebral disc is to absorb injuries and dissipate the compressive force applied to the spine by gravity. The discs convert this vertical pressure in a horizontal strength. The natural elasticity of the fibrous rings allows an increase in the disc's diameter. This small amount of movement in the horizontal plane helps in the spine's stability.

Degenerative changes in the spine often cause the loss of normal structure and/or function. The intervertebral disc is one structure prone to the degenerative changes associated with wear and tear aging, even misuse (e.g. smoking). This degeneration leads to a reduction in the thickness and compressibility of the disc and can lead to very severe sensations of pain.

When degeneration reaches an advanced stage, it is often necessary to remove the natural intervertebral disc and to replace it. If the entire disc is removed, spinal column instability may warrant fusion. Spinal fusion, the conventional treatment for a degenerated disc, may be combined with spinal instrumentation, the use of medically designed hardware (e.g. screws, cages). Fusion involves placing bone graft from the patient's pelvic bone and inserting metal rods or cages to stabilize the spine. Although spinal fusion can relieve pain by eliminating movement at the motion segment, it often decreases the patient's functional range of motion and may increase stress to the adjacent discs and facet joints. Spinal fusion naturally presents the drawback, particularly if applied to several vertebrae, of considerably limiting the patient's ability to move.

Alternatively, the disc may be completely replaced with an artificial disc, which is mounted between the vertebrae and which, ideally, conserves for the patient all of the relative mobility between the vertebrae, or at least a large fraction thereof.

### SUMMARY OF THE INVENTION

The invention provides an improved method of treating a degenerative spinal disorder comprising stabilizing an intervertebral disc and promoting a healing environment by inhibiting the inflammatory process. The degenerative spinal disorder is treated by implanting a spinal stabilization device, e.g., an extradiscal or intradiscal device, in combination with administration of an agent, e.g., an anti-inflammatory agent, a growth factor, an anti-angiogenesis agent, or an anti-enzymatic agent. In one embodiment the intradiscal device comprises a biocompatible polymer, such as a hydrogel material.

The invention includes a method of treating a degenerative intervertebral disc disorder comprising implanting a disc stabilization device into a subject suffering from the disorder and administering at least one therapeutic agent which promotes healing of the disc to the subject such that treatment of the disorder occurs, wherein the agent is selected from the group consisting of an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production.

The invention also includes a method of treating a degenerative spinal disorder comprising administering a therapeutic agent which promotes healing of the disc to a subject who has a disc stabilization device, wherein the agent is selected from the group consisting of an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production.

The invention provides a method of improving the disc quality of a damaged or degenerated intervertebral disc in a subject suffering from a degenerative intervertebral disc disorder comprising decreasing the load of the disc through a load bearing disc stabilization device in the subject; and administering a therapeutic agent to the subject, wherein the agent is selected from the group consisting an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production.

The invention also provides a method of promoting a healing environment for the treatment of a damaged or degenerative disc in a subject comprising decreasing the load of the disc through a load bearing disc stabilization device in the subject; and administering a therapeutic agent to the subject, wherein then agent is selected from the group consisting an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production.

The invention also includes a method of treating degenerative disc disease comprising implanting an intradiscal stabilization device into a damaged or degenerated intervertebral disc of a subject suffering from a degenerative spinal disorder and administering a therapeutic agent which inhibits the inflammatory process associated with the damaged or degenerated disc.

In one embodiment of the invention, the disc stabilization device is load bearing. In another embodiment, the disc stabilization device is not load bearing.

In one embodiment of the invention, the disc stabilization device is an extradiscal stabilization device, such as an intervertebral device (Wallis system).

The extradiscal device may be an interspinous process-based device or a pedicle screw-based device. In one embodiment, the interspinous process-based device is selected from the group consisting of an interspinous spacer, an interspinous process decompression (IPD) device, and a U-shaped interspinal device.

In one embodiment, the interspinous process-based device comprises an elastically, deformable wedge which is inserted between two spinous processes and has two lateral walls and two opposite grooves in which the spinous processes engage. In one embodiment, the intervertebral implant further comprises a fixing tie. In another embodiment, the intervertebral implant further comprises a fixing tie for retaining the spinous processes in the grooves; a removable self-locking fixing member having first connecting means and through which the tie can slide when it moves in translation in a first direction, the self-locking fixing member being adapted to immobilize the tie against movement in translation in a second direction opposite to the first direction; and at least one of the lateral wall so the first direction causing the spinous process to be clamped in the groove and the tie to be immobilized against movement in; and at least one of the lateral walls of the wedge includes a second connecting means to cooperate with the first connecting means to connect the removable self-locking fixing member to the lateral wall, a movement of the free end of the tie to move the tie in translation in the first direction causing the spinous processes to be clamped in the groove and the tie to be immobilized against movement in translation relative to the block in the second direction.

In one embodiment of the invention, the extradiscal device is coupled with a biocompatible hydrogel. The hydrogel may comprise the therapeutic agent.

In one embodiment of the invention, the disc stabilization device is an intradiscal stabilization device. In one embodiment, the intradiscal implant comprises hydrogel. In another embodiment, the intradiscal implant is an artificial nucleus pulposus which supports or replaces the existing nucleus pulposus, or a portion thereof, of the intervertebral disc. In an additional embodiment, the artificial nucleus pulposus contains a load bearing polymer. In still another embodiment, the artificial nucleus pulposus contains a non-load bearing polymer. In a further embodiment, the artificial nucleus pulposus comprises a biocompatible hydrogel. In still another embodiment, the artificial nucleus pulposus comprises a biomaterial selected from the group consisting of collagen type I, chytosan, fibrin, alginate, hyaluronate, cellulose, glycolide (PGA), polylactide (PLA) foam, and polyacrilonitril.

In one embodiment, the agent which inhibits pro-inflammatory cytokines comprises a TNFα inhibitor or an anti-IL1 inhibitor. The anti-TNFα inhibitor may be an antibody, or antigen binding portion thereof, including, but not limited to, a human antibody. The anti-IL1 agent may be an antibody, or antigen binding portion thereof. In one embodiment, the anti-TNFα a antibody is an isolated human antibody, or an antigen-binding portion thereof. In one embodiment, the human antibody, or an antigen-binding portion thereof, dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. In another embodiment, the anti-TNFα antibody is an isolated human antibody, or an antigen-binding portion thereof, with the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12. In still another embodiment, the anti-TNFα antibody is an isolated human antibody, or an antigen-binding portion thereof, with a light chain variable region (LCVR) comprising the amino acid sequence of SEQ ID NO: 1 and a heavy chain variable region (HCVR) comprising the amino acid sequence of SEQ ID NO: 2. In one embodiment, the anti-TNFα antibody is Humira (D2E7; adalimumab), golimumab, or Remicade (infliximab). In another embodiment, the anti-TNFα inhibitor is a fusion protein. In one embodiment, the fusion protein is Enbrel (etanercept).

In one embodiment, the growth factor is a member of the TGFα superfamily, such as, but not limited to, BMP2 or BMP7.

In one embodiment, the agent which inhibits angiogenesis inhibits VEGF.

In one embodiment, the anti-enzymatic agent is an anti-aggrecanase, such as but not limited to an anti-aggrecanase agent is directed to ADAMTS5. In still another embodiment, the anti-enzymatic agent is an anti-metalloproteinase.

In one embodiment of the invention, the therapeutic agent is delivered using a delivery means selected from the group consisting of direct injection, implantation with a drug delivery implant, and gene therapy.

In another embodiment, the invention further comprises administering an autologous cell or a regenerative growth factor which restores or improves disc tissue. In one embodiment, the cell which may be administered is selected from the group consisting of a chondrocyte, a mesenchymal stem cell, and an adipocytic stem cell.

In one embodiment, the chondrocytes are obtained from at least one source selected from the group consisting of the degenerative disc, an intact non-degenerative disc, and a non-disc cartilaginous source.

In one embodiment, the method of the invention is used to treat degenerative disc disease.

The invention also provides an intradiscal implant for promoting healing of a damaged or degenerated intervertebral disc comprising a biocompatible hydrogel and a therapeutic agent which promotes healing of the disc, wherein the therapeutic agent is selected from the group consisting of an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production. In one embodiment of the invention, the hydrogel is load bearing. In another embodiment, the hydrogel is non-load bearing polymer. In one embodiment, the intradiscal device comprises an artificial nucleus pulposus. In still another embodiment, the intradiscal device is designed to be injected in the disc.

In one embodiment, the agent which inhibits pro-inflammatory cytokines comprises a TNFα inhibitor or an anti-IL1 inhibitor. In another embodiment, the anti-TNFα inhibitor is an antibody, or antigen binding portion thereof. In still another embodiment, the anti-TNFα antibody is a an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. In a further embodiment, the anti-TNFα antibody is an isolated human antibody, or an antigen-binding portion thereof, with the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12. In yet another embodiment, the anti-TNFα antibody is an isolated human antibody, or an antigen-binding portion thereof, with a light chain variable region (LCVR) comprising the amino acid sequence of SEQ ID NO: 1 and a heavy chain variable region (HCVR) comprising the amino acid sequence of SEQ ID NO: 2. In a further embodiment, the anti-TNFα antibody is Humira (D2E7; adalimumab) or Remicade (infliximab). In one embodiment of the invention, the anti-TNFα inhibitor is Enbrel (etanercept).

In one embodiment, the invention provides a device implant comprising the anti-IL1 agent is an antibody, or antigen binding portion thereof.

In another embodiment, the intradiscal device of the invention comprises a growth factor which is a member of the TGFβ superfamily, such as, but not limited to, BMP2 or BMP7.

In yet another embodiment, the intradiscal implant of the invention comprises an agent which inhibits VEGF and inhibits angiogenesis.

In a further embodiment of the invention, the anti-enzymatic agent is an anti-aggrecanase or an anti-metalloproteinase. In one embodiment, the anti-aggrecanase agent is directed to ADAMTS5.

In one embodiment, the therapeutic agent is delivered using a delivery means selected from the group consisting of direct injection, implantation with a drug delivery implant, and gene therapy.

### DETAILED DESCRIPTION OF THE INVENTION

The invention provides a combination treatment for improving disc quality and promoting disc healing in a subject suffering from a degenerative spinal disorder. The combination method of the invention includes implanting a spinal implant, i.e., a disc stabilization implant, which increases stabilization of the vertebral column, and administering a therapeutic agent, such as an anti-inflammatory agent, a growth factor, an anti-angiogenesis agent, an anti-enzymatic agent, or a combination thereof. In addition, the invention provides an intradiscal implant, e.g., hydrogel, in which a therapeutic agent is embedded for the treatment of a degenerative spinal disorder.

I. Definitions

In order that the present invention may be more readily understood, certain terms are first defined. These terms are also discussed in further detail below in sections I-VII.

The term “disc,” “intervertebral disc,” or “vertebral disc,” used interchangeably herein, includes a disc which is interposed between the vertebrae of the vertebral column.

A “degenerated disc” includes a disc which has decreased flexibility, elasticity, and/or shock absorbing characteristics relative to a normal, healthy disc of the subject. Disc degeneration is a continuous process and, therefore, includes a wide range of degenerative states, for example from an intact disc containing small fissures in the annulus fibrosus (mild) to a ruptured disc (severe).

The term “load bearing” describes a device or implant which is capable of bearing a structural load. In one embodiment of the invention, the disc stabilization implant is load bearing and removes some, if not all, of the load of an intervertebral disc, which normally acts as a shock absorbing system for the spine.

A “disc stabilization implant” or a “disc stabilization device,” used interchangeably herein, includes any extradiscal or intradiscal dynamic stabilization system which helps stabilize the motion, extension, and/or flexion of the spine. In a preferred embodiment, the disc stabilization implant acts through a nonfusion method, wherein the disc stabilization implant does not include disc fusion.

An “extradiscal stabilization implant” or an “extradiscal stabilization device,” used interchangeably herein, includes an implant which is placed at a location outside but closely adjacent to a vertebral disc. The extradiscal stabilization device acts as a supplemental support device to the spine region containing the degenerative disc. In one embodiment, an extradiscal stabilization device is attached to the spinuous processes of the spine while leaving the intervertebral disc intact, i.e., a pedicle screw-based device, wherein screws are placed at two or more consecutive spine segments between the pedicles with a spacer, such as, for example, a rod, used to connect the screws.

In another embodiment, an extradiscal stabilization device placed between the spinous processes outside of the damaged disc, i.e., an interspinous process-based device. An example of an interspinous process-based device is an interspinous spacer, which includes, but is not limited to, a wedge which is inserted between the spinous processes and extends to the posterior part of two consecutive vertebrae, limiting movement towards each of the two vertebrae. In one embodiment, the interspinous spacer used in the method of the invention is the Wallis implant system (Spine Next; Abbott Spine), as described in U.S. application Ser. Nos. 10/332,798 and 10/471,213, U.S. Pat. No. 6,761,720, as well as FR 0300555, FR 0405611, and WO 04/073532, each of which is incorporated by reference herein.

An “intradiscal stabilization implant” or “intradiscal stabilization device,” includes any device or implant which can be placed within the disc space, on the disc, or as a replacement to the disc, or a portion thereof. An intradiscal implant may change size or shape within the disc space once implanted. In one embodiment of the invention, the intradiscal implant comprises hydrogel.

The term “hydrogel,” is described in the art and/or includes a gel comprising hydrophilic polymers in which water is present, usually in a large amount. In one embodiment, hydrogel includes a water-insoluble, water-containing, i.e., hydrophilic, poly- or monomeric material. Hydrogel materials are beneficial in the treatment of a degenerated disc as they can absorb water and have similar characteristics to the nucleus pulposus of a natural disc. In one embodiment of the invention, hydrogel is used as an intradiscal device for treating degenerative disc disease. In another embodiment, hydrogel is used in combination with an extradiscal device. The invention also includes compositions and methods of using hydrogel which acts as a carrier for a therapeutic agent. Hydrogel materials and uses thereof are described in more detail below, in section I.

The term “antibody”, as used herein, includes an immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or simply “antibody portion”), as used herein, includes one or more fragments of an antibody that retain the ability to specifically bind to an antigen (e.g., hTNFα). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883).

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Such chimeric, humanized, human, and dual specific antibodies can be produced by recombinant DNA techniques known in the art, for example using methods described in PCT

International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539.

An “isolated antibody”, as used herein, is intended to include an antibody that is substantially free of other antibodies having different antigenic specificities (e.g., an isolated antibody that specifically binds hTNFα is substantially free of antibodies that specifically bind antigens other than hTNFα). An isolated antibody that specifically binds an antigen, e.g., hTNFα, may, however, have cross-reactivity to other antigens, such as TNFα molecules, from other species. Moreover, an isolated antibody may be substantially free of other cellular material and/or chemicals.

A “neutralizing antibody”, as used herein, is intended to include an antibody whose binding to an inflammatory cytokine, such as hTNFα, results in inhibition of the biological activity of the inflammatory cytokine. This inhibition of the biological activity of the inflammatory cytokine can be assessed by measuring one or more indicators of biological activity associated with the inflammatory cytokine, such as, for example, hTNFα-induced cytotoxicity (either in vitro or in vivo), hTNFα-induced cellular activation and hTNFα binding to hTNFα receptors. These indicators of the inflammatory cytokine, such as hTNFα biological activity, can be assessed by one or more of several standard in vitro or in vivo assays known in the art. Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

II. Disc Stabilization Device

The invention provides a method of treating a degenerative intervertebral disc disorder comprising implanting a disc stabilization device into a subject with a disc disorder and administering at least one therapeutic agent which promotes healing of the disc. The invention also provides a method of improving the disc quality of a damaged or degenerated intervertebral disc in a subject suffering from a degenerative intervertebral disc disorder comprising decreasing the load of the disc through a load bearing disc stabilization implant in the subject; and administering a therapeutic agent to the subject. The invention further provides a method of promoting a healing environment for the treatment of a damaged or degenerative disc in a subject comprising decreasing the load of the disc through a load bearing disc stabilization implant in the subject; and administering a therapeutic agent to the subject.

The methods and compositions of the invention provide an improvement over standard techniques for treating disc disorders by promoting a stable physical and biological environment which promotes the healing of the damaged or degenerated disc without fusing discs together. The methods and compositions of the invention seek to inhibit the inflammatory process which occurs in a damaged or degenerated disc and can lead to further damage. The methods and compositions of the invention stabilize both the biological and physical environments of the disc to promote healing of the disc by combining administration of a therapeutic agent, such as an anti-inflammatory agent, in combination with the implantation of a spinal disc stabilization device which increases the stabilization of the vertebral column. Thus, the invention provides a combination treatment for improving disc quality and promoting disc healing in a subject suffering from a degenerative spinal disorder.

In one embodiment of the invention, the disc stabilization device is load-bearing. The intervertebral joint exhibits both elastic and viscous behavior. During the normal application of a load to the disc there will be an immediate distortion or deformation of the disc, often referred to as “instantaneous deformation”. Thus, a damaged or degenerated disc may not accommodate the load placed on it. The invention provides an improved method of treating degenerative spinal disorders in a subject comprising both reducing the load which the damaged or degenerated vertebral disc bears in combination with reducing inflammation. The combination of reducing the forces exerted on the disc and the reduction of the inflammation helps to promote healing of the disc. The methods and compositions of the invention allow the disc to heal, and are an improvement over those in the art as the invention does not include disc fusion or complete replacement, each of which may result in limited movement. The method and compositions of the invention provide a means by which the load which is placed on a damaged or degenerated disc is decreased by off-setting the load with a disc stabilization device, i.e., a load bearing intradiscal or extradiscal device.

Examples of disc stabilization devices include extradiscal devices which are external to the degenerated disc and intradiscal devices which act within or replace the damaged disc.

A. Extradiscal Stabilization Device

In one embodiment of the invention, degenerative disc disease is treated by implanting an extradiscal stabilization device in a subject, which provides support to the degenerated disc. Examples of extradiscal stabilization devices include, but are not limited to, a pedicle screw-based device and an interspinous process device (or intervertebral implant).

An extradiscal stabilization device may be made of any type of biocompatible material, including polyether ether ketone and plastics. Extradiscal stabilization devices may also be coupled with a hydrogel, which may or may not contain at least one therapeutic agent, such as an agent which inhibits pro-inflammatory cytokines; an anti-enzymatic agent which inhibits degradation of the extracellular matrix of the disc; an agent which inhibits angiogenesis in the disc; a growth factor which promotes extracellular matrix production; or a combination thereof.

1. Interspinous Process-Based Device

In one embodiment, the extradiscal stabilization devices is an interspinous process-based device (also referred to as an intervertebral implant), which is placed between the spinal processes outside of the disc to provide support for the disc and allow the disc to heal. An interspinous process-based device can compensate for a vertebral disk when the disk is deficient and in particular it limits the extent to which the posterior portions of two vertebrae can move towards each other when the spine is extended. An interspinous process-based device is implanted between the spinous processes and distracts the spinous processes which may move closer together than normally permitted due to a degenerated disc. Thus, the interspinous process-based device reduces extension but allows flexion and rotation of the spine.

It is beneficial that the interspinous process-based device be fixed to the vertebrae in a manner that is sufficiently rigid to keep it in position regardless of the movements of the spine. In addition, the interspinous process-based device should be sufficiently free relative to the same vertebrae to avoid excessively stiffening the vertebral column. Examples of interspinous process-based devices include, but are not limited, to an interspinous spacer and an interspinous process decompression device.

a. Interspinous Spacer

In one embodiment, the interspinous process-based device is an interspinous spacer (also referred to as a wedge or block) which is used to inhibit movement of the vertebrae towards each other in order to treat degenerative pathologies of the intervertebral disc which cause the vertebrae to move towards each other, possibly to the extent that they come into contact. When vertebrae move close enough to touch one another, they can pinch the roots of the nerves routed laterally between the vertebrae which is painful for the afflicted subject. One advantage of an interspinous spacer is that vertebral stability is achieved by preserving bone and ligament preservation without permanent bony fixation, i.e., there is no bony purchase or fixation.

An interspinous spacer may be fixed between the spinous processes of two consecutive vertebrae that come into contact on movement of the spine, using appropriate fixing means. The interspinous spacer is intended to establish a specified space between the vertebrae on either side of the intervertebral disc being treated, thus limiting the mechanical stresses on that disc.

The Wallis system (Abbott Spine) may be used as an interspinous spacer (or intervertebral implant), as described in U.S. Pat. Nos. 6,946,000 and 6,761,720, as well as US Appln. No. 2004/0117017 (application Ser. No. 10/471,213), each of which is incorporated by reference herein.

The Wallis system comprises an interspinous process spacer (also referred to as a wedge or block) and stabilizing bands or ties which are passed around adjacent spinous processes. The intervertebral device is fastened through the wrapping of bands around the bone. The Wallis system does not alter the vertebrae like other procedures that use screws or similar fastening methods, as the facet joints and pedicles are not disturbed. The Wallis procedure is advantageous because it is less invasive than other surgical procedures that use instrumentation. The Wallis system relieves pain by increasing the rigidity of the unstable segment, while preserving its mobility. The design and materials minimize the need for bony resection and avoid any concentration of constraint on the bone. The anatomic design of the Wallis system includes notches that fit the physiological shape of the spinous processes. The Wallis system also provides optimization of the surface of contact, using a flat band for best spread of constraints in contact with the bone. Finally, the Wallis system provides elasticity by employing a spacer and clips made of PEEK for mechanical properties close to those of bone and openings in the spacer for maximum reduction of the implant rigidity.

In one embodiment, the interspinous spacer comprises a central part and two end parts, with each end part having a channel bordered by two wings, said channel being suitable for receiving the spinous apophysis of one of the vertebrae adjacent to the disc being treated. The interspinous spacer may also comprise a spacer having two opposite notches for receiving two spinous processes of two vertebrae, each notch defining a bottom and two flanges, each having an inside wall; a tie for holding said spacer to said spinous processes, said tie being constituted by at least one strap having first and second ends and a portion surrounding a portion of a surface of a spinous process opposite from the bottom of a notch; first fixing means formed in at least one of said flanges to fix the first end of said strap; and self-locking second fixing means formed in at least another one of said flanges, the second end of said strap being inserted through said self-locking second fixing means and then pulled to hold said strap in position, thereby securing said spacer to said spinous processes. Additional details and examples of such interspinous spacer devices are described in U.S. Pat. No. 6,761,720, which is incorporated by reference herein.

In one embodiment, the interspinous spacer comprises a deformable wedge (also referred to as an intervertebral implant with a deformable wedge) made from a rigid, biocompatible material obtained by polymerization. The wedge may have a central opening between two grooves, wherein the central opening passes completely through the wedge along an axis substantially parallel to the axes of the grooves, and the volume of the central opening is from 10% to 30% of the total volume of the wedge, which renders the wedge elastically deformable. An important feature of the deformable wedge is that the wedge can deform elastically without being made excessively fragile. The wedge constitutes an obstacle to movement of the vertebrae towards each other. However, the forces exerted by the wedge on the spinous processes are proportional to the relative movements of the two vertebrae, since the wedge is elastically deformable, which reproduces normal or virtually normal physiological conditions of relative movement of the vertebrae. The deformable wedge may also include a fixing band (which may be pre-mounted) adapted to retain the wedge between the spinous processes. The wedge may have a fixing means for connecting the band to the wedge and a self-locking fixing means in its lateral walls which are adapted to receive the fixing band in order to immobilize it relative to the wedge. Additional details and examples of such interspinous spacer devices are described in U.S. Pat. No. 6,946,000 and WO 02/051326, which are both incorporated by reference herein.

The interspinous spacer may be fixed into place between the spinous processes of two vertebrae using a means of attaching the wedge to the vertebrae, i.e. a tie for holding the spacer between two vertebrae. In one embodiment, the interspinous spacer is a self locking fixable spacer, wherein a block is inserted between then spinous processes and fixed in place with a tie. The tie may be attached at one end to the block with a free end which is used to surround at least one spinous process. In one embodiment the interspinous spacer comprises a block having two opposite sides in the direction of a longitudinal axis, each side being provided with a groove adapted to receive the spinous processes, wherein the block further has two lateral walls. The interspinous spacer may further comprise a fixing tie for retaining the spinous processes in the grooves, the fixing tie having a first end adapted to be connected to the block and a second free end, where the tie is adapted to surround at least one spinous process. The interspinous spacer may further comprise a removable self-locking fixing member having a first connecting means and through which the tie can slide when it moves in translation in a first direction, wherein the self-locking fixing member is adapted to immobilize the tie against movement in translation in a second direction opposite the first direction. Furthermore, the interspinous spacer may comprise at least one of the lateral walls of the block including a second connecting means to cooperate with the first connecting means to connect the removable self-locking fixing member to the lateral wall, a movement of the free end of the tie to move the tie in translation in the first direction causing the spinous process to be clamped in the groove and the tie to be immobilized against movement in translation relative to the block in the second direction. Additional details and examples of interspinous spacer comprising a block/tie system are described in US Appln. No. 10/471,213 (US 2004/0117017) and EP Patent 1367954, each of which is incorporated by reference herein.

The interspinous spacer may also be specific to the lumbrosacral articulation, as described in FR 0310063 (AU4268404 and WO 05/020860; Spine Next), the entirety of which is incorporated by reference herein. The inventive implant comprises a wedge which is disposed between a fifth lumbar vertebra (L5) and the sacral vertebra (S1) which is articulated to L5. The upper face of the body of the wedge comprises a groove which is oriented along the mid-plane (M) of said wedge and which can receive the spinous process of the aforementioned lumbar vertebra (L5). According to the invention, a longitudinal housing, which is positioned orthogonally to the aforementioned groove, is provided on the lower face and is designed to receive the upper part of the sacral vertebra (S1). The longitudinal housing is defined by an extension and a tab having a greater width than that of the body of the wedge. Moreover, the section of the housing, along the mid-plane (M) of the wedge, takes the general form of a U which is inclined in relation to the base of the groove.

Examples of other intervertebral implants which can be used in the methods of the invention are described in WO 01/28442; FR 0212397; WO 02/07621; WO 02/07622, WO 04/073532, and WO 05/020860, each of which is expressly incorporated by reference herein.

b. Interspinous Process Decompression (IPD) Device (X-Stop®)

In one embodiment, the interspinous process-based device is an interspinous process decompression (IPD) device which is placed between the spinal processes of the symptomatic disc levels, such that the IPD device distracts the space between the spinal processes and maintains it in a slightly flexed position. The IPD device allows the recipient to resume a normal posture rather than flex their spine to gain relief of symptoms.

The IPD device comprises a body having a spacer and an alignment track, a wing having an alignment tab to engage the alignment track on the body, and a fastening device that secures the wing to the body. In one embodiment, the IPD device comprises a main body made from a rigid, biocompatible material. The main body may have a tissue expander having the smallest tip then gradually increasing until the implant is substantially similar to the diameter of the main body. The tissue expander distracts the adjacent spinous processes to the diameter of the spacer. The insert may contain a cylindrical spacer which can swivel, making it self-aligning relative to the uneven surface of the spinous process. The insert may also contain an adjustable wing allowing for easy assembly and minimally invasive surgery. The oval spacer separates the spinous processes and restricts terminal extension movement at the symptomatic levels while allowing unrestricted movement of the remaining motion axes of the treated levels.

An example of an IPD device is X-Stop® (St. Francis Medical Technologies, Inc.), as described in U.S. Pat. No. 6,695,842 (assignee: St. Francis Medical Technologies, Inc.), incorporated by reference herein.

c. U-Shaped Interspinal Device

In one embodiment, the interspinous process-based device is a U-shaped interspinal device which comprises a substantially U-shaped body, providing for flexible positioning of the vertebrae in relation to one another. An example of a U-shaped interspinal device is the Inter-spinous U (Fixano).

The U-shaped interspinal device relieves symptoms of degenerative disk disease by stabilizing the affected spinal segment by permitting flexion and extension of the spinal column on either side of a neutral position corresponding to the substantially parallel position of the two branches of its U-shaped body. The U-shaped interspinal device may be placed in the interspinal space, with the central portion of its body providing flexible positioning. The U-shaped interspinal device is inserted into the interspinal space without requiring special work at the site receiving the implant.

In one embodiment, the U-shaped interspinal device is made from a rigid, biocompatible material. The U-shaped body may have an elastic flexibility in the area of its central portion which provides for flexible positioning of the vertebrae in relation to one another. The elastic flexibility allows a flexion and extension of the spinal column. The U-shaped interspinal device may also have two pairs of brackets projecting form the outer face of the two branches of the body. The brackets provide stirrups for receiving the spinous processes.

In one embodiment, the U-shaped interspinal vertebral device comprises a U-shaped body having a central portion and two branches, wherein at least the central portion is elastically flexible; two pairs of brackets, wherein each pair of brackets projects from an outer face of one of the two branches, and wherein each pair of brackets comprises a stirrup for receiving a spinous process of a vertebrae; and

means wherein said brackets includes for attaching the brackets to spinous processes of the vertebrae. Examples of such U-shaped interspinal devices are described in U.S. Pat. No. 5,645,599 (Assignee, Fixano), incorporated by reference herein.

2. Pedicle Screw-Based Device

The extradiscal stabilization device may be pedicle screw-based device, wherein screws are inserted into the spinous pedicles and are used to affix an interspinous spacer, such as a rod or plate, to the spine. The pedicle screws may be designed to have rotational capabilities, providing increased mobility to the recipient.

a. Dynamic External Spacer Stabilization System (Dynesys®)

The pedicle screw-based system may be a dynamic external spacer stabilization system comprising an external spacer attached to the spine through pedicle screws. An example of a dynamic external spacer stabilization system is Dynesys® (Zimmer Spine; see Stoll et al. European Spine Journal 11 Suppl 2: S170-8, 2002; Schmoelz et al, J of spinal disorder & techniques 16(4):418-23, 2003). The Dynesys device uses a polycarburethane spacer between the screws to maintain the distance between the heads of two corresponding pedicle screws and, hence, adjacent vertebrae in which the screws are fixed. Dynesys comprises external spacers which may be made of surgical polyurethane tubing surrounding a polyethylene cord. The cord and spacers provide a dynamic push/pull relationship that stabilizes the affected vertebrae. Dynesys attaches to both sides of the vertebrae in the affected area of the spine, stabilizing the spine without fusing and preserving its natural curve, which allows a greater degree of mobility and reduced pain. Once the external spacer devices are attached bilaterally to the affected segments, the dynamic push-pull relationship between spacer and cord stabilizes the joints, keeping the vertebrae in a more natural position.

b. Flexible Jointed Rod (ISOBAR TTL)

In one embodiment, the pedicle screw-based system is a flexible jointed rod attached using a pedicle screw system. The rod may be made flexible by a small joint in the rod (a dampener) which provides additional degrees of motion. The joint provides enough flexibility to effectively absorb flexion and extension stresses and reduce the rate of adjacent level deterioration around the fused levels.

In one embodiment, the flexible jointed rod is attached using both pedicle screws and auto-stabilizing thoracic hooks arranged to clamp at least one vertebra, thus providing stabilization of bone anchoring by means of hooks. The pedicle screw and hook-based implant is based upon the principle of posterior fixation by the use of pedicular screws and hooks. The pedicle screw implant also includes a free connection means between the hook and the counterhook enabling them to be moved freely by means of an elongate member or a finger. The sliding of the finger of the hook enables the hook and the counterhook to clamp a vertebra to a greater or lesser extent by acting on opposite sides of the vertebra.

An example of a flexible jointed rod which can be used in the invention is the ISOBAR TTL system (Scient'x). Additional detail and examples of flexible jointed rods are described in U.S. Pat. No. 6,241,730 and U.S. Pat. No. 6,387,097 (Scient'x), incorporated by reference herein.

c. Helical Rod (AccuFlex™ Rod)

In another embodiment, the pedicle-screw based device is a helical rod, which comprises one or more flexible elements with tubular structures having openings or slits, which may form helical patterns on the tubular structures. A helical rod provides load sharing either as an enhancement to a fusion device or as a motion-preserving non-fusion device. The flexible elements of the helical rod may be conformable to the natural spinal movement. The system also may have a locking mechanism that secures one or more flexible elements in a rigid configuration if required.

The helical rod may comprise a first flexible element having a first and second end, wherein at least a portion of the first flexible element comprises a first tubular structure having at least a first slit formed therein; a second flexible element disposed within the first flexible element, wherein at least a portion of the second flexible element comprises a second tubular structure having at least a second slit formed therein; a first fastener connected to the first end of the first flexible element; and a second fastener connected to the second end of the first flexible element.

The helical rod may also comprise a tubular structure that has at least one slit formed in it, and a second flexible element disposed within the first flexible element. The second flexible element also has a tubular structure with at least on slit formed in it. The slit or slits formed on either or both of the tubular structures may form a generally helical pattern around a longitudinal axis of the tubular structure. An important feature of the system is that the first and second spiral patterns travel in opposite directions along the first and second rods, allowing greater flexibility in balancing a response to torsional forces applied to the assembly of rods. The flexible element provides stability, strength, flexibility, and resistance.

An example of a helical rod which can be used in the invention is the AccuFlex™ rod (Globus Medical). Additional detail and examples of such helical rods are described in U.S. Pat. Nos. 6,986,771 and 6,989,011 (Globus Medical), incorporated by reference herein.

d. Total Posterior Spine System: (T.O.P.S.)

The invention also includes use of a total posterior spine system (TOPS) to stabilize but not fuse the affected vertebral level using a mobile posterior device. The device is affixed to the spine via at least four pedicle screws using a standard posterior surgical approach. Additional detail and examples regarding TOPS can be found in WO 05/044152 (Impliant Inc), incorporated by reference herein.

e. Device for Intervertebral Assisted Motion: (DIAM)

The invention also includes use of an interspinous process-based device which is a device for intervertebral assisted motion (DIAM™; Medtronic). The DIAM™ device relieves symptoms of degenerative disc disease by acting primarily as a shock absorber that fits in between two vertebrae, wherein the device allows for optimal mechanical behavior in compression and flexion. The DIAM™ device is inserted where part of the interspinous ligament is removed and fastened between two vertebrae using ligatures that are passed around the upper and lower spinous processes. The DIAM™ device is a silicone interspinal cushioning device covered with polyethylene. The shape of the implant provides stability by means of extended wings bracing the spinous processes. The DIAM™ function is to reduce the intradiscal pressure, to enable the ligament structure functional again and control the excess movement of the degenerated segment.

B. Intradiscal Stabilization Device

In one embodiment of the invention, degenerative disc disease is treated by implanting an intradiscal stabilization device or implant in a subject. An example of an intradiscal stabilization device includes, but is not limited to, a type of disc stabilization implant which acts within or as a replacement to the natural disc or a portion thereof. The intradiscal stabilization device may replace any region of the disc, including the annulus fibrosus, nucleus pulposus, or portions thereof. In addition, extradiscal devices may be coupled with intradiscal devices, such as hydrogel, described in more detail below. The invention also describes an intradiscal treatment comprising administering a biomaterial comprising a therapeutic agent. In one embodiment, the intradiscal treatment comprises a hydrogel comprising an anti-TNFa antibody, e.g., Humira.

Artificial Nucleus Pulposus

The intradiscal stabilization implant which is used in the method of the invention to stabilize the disc may be an artificial (or prosthetic) nucleus pulposus. An artificial nucleus pulposus implant may contain a load bearing or a non-load bearing polymer. Ideally, the artificial nucleus pulposus implant replicates both the biologic, e.g., hydrophilic, and biomechanical, e.g., flexibility and the ability to withstand force, characteristics of a healthy nucleus, thereby restoring the natural movement of the spine.

An artificial nucleus pulposus serves to support an intervertebral disc in the same manner as the natural disc and, therefore, has similar characteristics, i.e, good mechanical strength which permits them to withstand the load on the disc and/or restore the normal space between the vertebral bodies. The artificial nucleus pulposus used in the invention may be made of any biocompatible material with these properties. An example of a material which can be used as an artificial nucleus pulposus includes xerogel which is capable of anisotopic swelling, see U.S. Pat. No. 6,264,694, incorporated by reference herein.

In one embodiment, the artificial nucleus pulposus comprises a biocompatible hydrogel material, as described below. Examples of hydrogels which can be used as an artificial nucleus pulposus are described in U.S. Pat. No. 5,047,055; U.S. Pat. No. 5,976,186; and U.S. Pat. No. 5,534,028; and U.S. Pat. No. 6,726,721 each of which are incorporated by reference in the entirety herein. Alternatively, an artificial nucleus pulposus may be made from a biomaterial such as, but not limited to, collagen type I, chytosan, fibrin, alginate, hyaluronate, cellulose, glycolide (PGA), polylactide (PLA) foam, and polyacrilonitril.

Examples of various types of artificial nucleus pulposus implants include, but are not limited to, expandable wafers (see U.S. Pat. No. 6,764,514, incorporated in its entirety by reference herein); an artificial nucleus pulposus comprising an expandable implant comprising an inflatable membrane with an internal chamber which has a self-sealing fill valve (see US Patent Appln. 2003/0033017, incorporated by reference herein); and a disc nucleus pulposus implant comprising a load bearing elastic body sized for introduction into an intervertebral disc space, wherein the body of the implant is surrounded by a resorbable shell (see US Patent Appln. No. 2002/0026244, incorporated by reference herein). Examples of methods of administering an artificial nucleus pulposus into a disc are described in U.S. Pat. No. 6,733,505, incorporated by reference herein.

Prior to implantation, the natural nucleus pulposus, or a portion thereof, may be removed to accommodate the artificial pulposus. Depending on the extent of degeneration of the disc, however, removal of the natural pulposus may not be necessary. The natural nucleus pulposus may be removed using standard techniques (enzymatically (chymopapain) or with the aid of a laser, suction device, shaver, or other surgical instrument). If the nucleus is removed the hole in the annulus should be small and sealed to prevent the in growth of vascular tissue.

III. Hydrogel

Hydrogel may also be used in the methods and compositions of the invention. In one embodiment, hydrogel is used in combination with an extradiscal device to provide a means for delivering a therapeutic agent, including, but not limited to, adalimumab. In another embodiment, hydrogel is used as an intradiscal stabilization device to provide support for a degenerated disc.

Hydrogel is a material remains intact in water that is a material which can shrink or swell and still remain intact without dissolving. Hydrogel allows for physiologic tension adjustment since it is a material which can change size based upon imbibing fluid and pressure on the hydrogel.

Hydrogels suitable for use in the present invention include water-containing gels, i.e., polymers characterized by hydrophilicity and insolubility in water. (See, for instance, “Hydrogels” in Concise Encyclopedia of Polymer Science and Engineering, Mark et al., eds. (Wiley and Sons), pp. 458-459 (1990), incorporated herein by reference. Although use of hydrogel is optional in the present invention, the inclusion of hydrogels can be highly advantageous since hydrogel has a number of desirable qualities.

Hydrogel is an elastomeric polymer which contains a large amount of water which acts as a plasticizer. Hydrogel may be dehydrated, such as when mechanical pressure is applied, and then rehydrated without changing the properties of the hydrogel. Types of hydrogel include, but are not limited to, polyvinyl alcohol (PVA) hydrogel, polyacrylonitrile hydrogel. lightly cross-linked polymers of 2-hydroxyethyl methacrylate, or copolymers and terpolymers made from the combination of the monomers of an N-vinyl monomer, (for example, N-vinyl-2-pyrrolidone (N-VP)), a hydroxy alkyl methacrylate ester, (for example, 2-hydroxylethyl methacrylate (HEMA)), an alkyl methacrylate (for example, methyl methacrylate (MMA)), an ethylenically unsaturated acid (for example, methacrylic acid (MA)) and an ethylenically unsaturated base (for example, N,N-diethylamino ethyl methacrylate (DEAEMA)), and hydrogel polyacrylonitrile. Hydrogel may also include any, i.e., one or more, of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

In one embodiment, the intradiscal implant used in the method of the invention may be made of a biocompatible hydrogel material. Hydrogel is advantageous for use as an intradiscal implant due to its unique characteristics which are similar to those of the native nucleus pulposus, i.e., hydrogel is flexible and can withstand forces. Thus, the hydrogel component of the intradiscal device can change height to balance the forces against the surrounding tissues. Animal experiments have shown that hydrogel intradiscal implants are effective as replacements for the nucleus pulposus (Allen et al. (2004) Spine 29: 515). In one embodiment, hydrogel is used to support or replace the nucleus pulposus of a deteriorated or deteriorating disc. Hydrogel may be injected into the damaged disc, where it assumes the shape of the interior cavity of the disc.

Hydrogel may also be used in other aspects of the invention in addition to intradiscal implants. By virtue of their hydrophilic, water-containing nature, hydrogels may be used to house viable cells, such as mesenchymal stem cells, and/or redistribute the load bearing and load transmission capabilities of the disc. Hydrogel may also be combined with the extradiscal device of the invention to support healing of a damaged disc.

In one embodiment, hydrogel which is used in the method of the invention is load-bearing. Hydrogel-based artificial nucleus pulposus has high mechanical strength and is able to withstand the body load and assist in the healing of the deteriorated disc. Load-bearing hydrogel may be used for implanting in the disc space after the removal of the degenerated or damaged nucleus pulposus of an intervertebral disc.

Hydrogel may also be used as a drug carrier, wherein it is used to administer the therapeutic agent used in the invention. Suitable hydrogels exhibit an optimal combination of carrier properties, including compatibility with the matrix polymer of choice and biocompatability. Other examples of types of hydrogels are described in U.S. Pat. No. 5,047,055; U.S. Pat. No. 5,976,186; U.S. Pat. No. 5,534,028; and U.S. Pat. No. 6,232,406 each of which are incorporated herein. Examples of methods of making hydrogel are also described in U.S. Pat. No. 6,451,922 and U.S. Pat. No. 6,232,406, each of which is incorporated herein.

IV. Therapeutic Agents

The invention describes a combination method of stabilizing an intervertebral disc through implantation of a disc stabilization device and administering at least one therapeutic agent to promote healing of the disc for the treatment of a degenerative intervertebral disc disorder. The therapeutic agent of the invention includes at least one of the following: an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production. An additional therapeutic agent may be used in conjunction with a disc stabilization device to treat a degenerative intervertebral disc disorder, wherein the disc stabilization device is implanted into a subject suffering from the disorder and the subject is administered at least one therapeutic agent which promotes healing of the disc to the subject such that treatment of the disorder occurs. An additional therapeutic agent may also be used in conjunction with a disc stabilization device to treat a degenerative spinal disorder, wherein a therapeutic agent which promotes healing of the disc is administered to a subject who has an implanted disc stabilization device. The additional therapeutic agent may be injected or implanted with a drug delivery device, such as but not limited to a hydrogel, into a subject have a degenerative intervertebral disc disorder. Examples of additional therapeutic agents which may be used in the invention are described in detail below:

A. Molecules that Inhibit Pro-Inflammatory Cytokines

The method of the invention includes administering an agent which inhibits pro-inflammatory cytokines and inhibits the inflammatory process in combination with a disc stabilization device for the treatment of a degenerative disc disorder. In one embodiment, anti-TNFα and anti-IL1 molecules are used to inhibit inflammation associated with the degenerative disc disorder, as described below.

The methods of the invention include administration of isolated human antibodies, or antigen-binding portions thereof, that bind to human TNFα with high affinity and a low off rate, and have a high neutralizing capacity. Preferably, the human antibodies of the invention are recombinant, neutralizing human anti-hTNFα antibodies. The most preferred recombinant, neutralizing antibody of the invention is referred to herein as D2E7, also referred to as HUMIRA® and adalimumab (the amino acid sequence of the D2E7 VL region is shown in SEQ ID NO: 1; the amino acid sequence of the D2E7 VH region is shown in SEQ ID NO: 2). The properties of D2E7 (adalimumab; HUMIRA®) have been described in Salfeld et al., U.S. Pat. No. 6,090,382, which is incorporated by reference herein.

In one embodiment, the treatment of the invention includes the administration of D2E7 antibodies and antibody portions, D2E7-related antibodies and antibody portions, and other human antibodies and antibody portions with equivalent properties to D2E7, such as high affinity binding to hTNFα with low dissociation kinetics and high neutralizing capacity. In one embodiment, the invention provides treatment with an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a K_{off} of 5×10⁻⁴ s⁻¹ or less, or even more preferably, with a K_{off} of 1×10⁻⁴ s⁻¹ or less. More preferably, the isolated human antibody, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to methods of treating a TNFα-related disorder in which the TNFα activity is detrimental by administering human antibodies that have slow dissociation kinetics for association with human TNFα and that have light and heavy chain CDR3 domains that structurally are identical to or related to those of D2E7. Position 9 of the D2E7 VL CDR3 can be occupied by Ala or Thr without substantially affecting the K_{off}. Accordingly, a consensus motif for the D2E7 VL CDR3 comprises the amino acid sequence: Q-R-Y-N-R-A-P-Y-(T/A) (SEQ ID NO: 3). Additionally, position 12 of the D2E7 VH CDR3 can be occupied by Tyr or Asn, without substantially affecting the K_{off}. Accordingly, a consensus motif for the D2E7 VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the K_{off}. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 appear to be critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382, incorporated by reference herein).

Accordingly, in another embodiment, the invention provides methods of treating a TNFα-related disorder by the administration of an isolated human antibody, or antigen-binding portion thereof. The antibody or antigen-binding portion thereof preferably contains the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a K_{off} of 5×10⁻⁴ s⁻¹ or less. Even more preferably, the antibody, or antigen-binding portion thereof, dissociates from human TNFαC with a K_{off} of 1×10⁻⁴ s⁻¹ or less.

In yet another embodiment, the invention provides methods of treating a TNFα-related disorder by the administration of an isolated human antibody, or antigen-binding portion thereof. The antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. Preferably, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). Even more preferably, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the V_{K}I human germline family, more preferably from the A20 human germline Vk gene and most preferably from the D2E7 VL framework sequences shown in FIGS. 1A and 1B of U.S. Pat. No. 6,090,382. The framework regions for VH preferably are from the V_{H}3 human germline family, more preferably from the DP-31 human germline VH gene and most preferably from the D2E7 VH framework sequences shown in FIGS. 2A and 2B of U.S. Pat. No. 6,090,382, incorporated by reference herein.

Accordingly, in another embodiment, the invention provides methods of treating a TNFα-related disorder by the administration of an isolated human antibody, or antigen-binding portion thereof. The antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention provides methods of treating a TNFα-related disorder in which the administration of an anti-TNFα antibody is beneficial administration of an isolated human antibody, or an antigen-binding portions thereof. The antibody or antigen-binding portion thereof preferably contains D2E7-related VL and VH CDR3 domains, for example, antibodies, or antigen-binding portions thereof, with a light chain variable region (LCVR) having a CDR3 domain comprising an amino acid sequence selected from the group consisting of SEQ ID NO: 3, SEQ ID NO: 1, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

In another embodiment, the TNFα inhibitor of the invention is a TNF fusion protein, e.g., etanercept (Enbrel®, Amgen; described in WO 91/03553 and WO 09/406,476, incorporated by reference herein). In another embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (Humira® Abbott Laboratories, a human anti-TNF mAb, described in U.S. Pat. No. 6,090,382 as D2E7), In another embodiment, the TNFα inhibitor is a recombinant TNF binding protein (r-TBP-I) (Serono).

In one embodiment, these methods include administration of isolated human antibodies, or antigen-binding portions thereof, which bind to human molecules, e.g., cytokines, associated with inflammation. Examples of such cytokines include IL-1, IL-6, TNF-α, and TGF-β. Interleukin-1 (IL-1) inhibitors may be from any protein capable of specifically preventing activation of cellular receptors to IL-1. Classes of interleukin-1 inhibitors include: interleukin-1 receptor antagonists such as IL-Ira, as described below; anti-IL-1 receptor monoclonal antibodies (e.g., EP 623674, incorporated by reference herein), incorporated by reference herein; IL-1 binding proteins such as soluble IL-1 receptors (e.g., U.S. Pat. No. 5,492,888, U.S. Pat. No. 5,488,032, and U.S. Pat. No. 5,464,937, U.S. Pat. No. 5,319,071, and U.S. Pat. No. 5,180,812, each of which is incorporated by reference herein); anti-IL-1 monoclonal antibodies (e.g., WO 9501997, WO 9402627, WO 9006371, U.S. Pat. No. 4,935,343, EP 364778, EP 267611 and EP 220063, each of which is incorporated by reference herein); IL-1 receptor accessory proteins (e.g., WO 96/23067, incorporated by reference herein), and other compounds and proteins which block in vivo synthesis or extracellular release of IL-1.

The promotion of healing of a degenerative disc in accordance with the methods and compositions of the invention may also be performed in combination with inhibitors, (for example an antibody to or antagonist of) of human cytokines or growth factors, including, for example, TNF, LT, IL-1, IL-2, IL-3, IL-4, IL-5, IL-6, IL-7, IL-8, IL-12, IL-15, IL-16, IL-18, IL-21, IL-23, interferons, EMAP-II, GM-CSF, FGF, and PDGF. The methods and compositions of the invention may also be combined with inhibitors, (for example an antibody to or antagonist of, of cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

B. Molecules that Inhibit Enzyme Degradation of the Extracellular Matrix

The invention includes administering an anti-enzymatic agent which inhibits degradation of the extracellular matrix of the disc in combination with a disc stabilization device for the treatment of a degenerative disc disorder. The invention provides a means by which a damaged disc can heal by promoting stabilization of the disc in combination with the benefits of an agent which prevents joint destruction, which often can result from the inflammatory process. Examples of anti-enzymatic agents which can be administered in combination with a disc stabilization implant include an anti-aggrecanase agent, an anti-metalloproteinase agent, or a combination thereof.

An anti-aggrecanase agent or aggrecanase inhibitor which can be used in the invention includes any agent which can cleave an aggrecan, a major proteoglycan found in cartilage extracellular matrix. Aggrecan functions to bring about osmotic swelling and maintains high levels of hydration in the cartilage extracellular matrix.

ADAMTS proteinases, belonging to the adamalysin subfamily of metalloproteinases, have been implicated in a variety of cellular events such as extracellular matrix breakdown, including the cleaving of aggrecan, morphogenesis, cell migration, angiogenesis, and ovulation. ADAMTS family members are implicated in mediating cartilage aggrecan loss. ADAMTS# gene symbols designate a subset of ADAM proteins that contain a thrombospondin (TS) motif. Members of the ADAMTS family share several distinct protein modules, including a propeptide region, a metalloproteinase domain, a disintegrin-like domain, and a thrombospondin type I (TS) motif. Individual members of this family differ in the number of C-terminal TS motifs, and some have unique C-terminal domains. An anti-aggrecanase agent includes any agent which inhibits the activity of members of the ADAMTS family, e.g., ADAMTS5 and ADAMTS4, including antibodies.

In one embodiment, the method of the invention includes administration of an agent which inhibits the activity of ADAMTS5, also referred to as ADMP-2 and ADAMTS11. ADAMTS5 encodes a disintegrin and metalloproteinase with thrombospondin motifs-5 (ADAMTS5) (see GenBank accession no. NM_{—}007038; Cross et al. (2005) Prostate 63: 269).

Metalloproteinases (MMPs) are members of a family of proteolytic enzymes (proteinases) characterized by a metal (usually zinc) prosthetic group which decompose the macromolecular components of the extracellular matrix, e.g., collagen, elastin (see Woessner, (1991) Faseb Journal 5:2145). Overexpression and activation of MMPs can result in numerous processes which involve the destruction and the remodeling of the matrix, including, for example, in an uncontrolled resorption of the extracellular matrix.

An anti-metalloproteinase (anti-MMP) agent or metalloproteinase inhibitor of the invention includes any agent which decreases, blocks, inhibits, abrogates, or interferes with MMP activity. Anti-MMP agents inhibit enzymatic activity associated with degradation of proteins of the extracellular matrix, including glycoproteins (fibronectin, laminin) and proteoglycans. Examples of anti-MMP agents which may be used to inhibit degradation of the disc include anti-collagenases which decompose fibrillar collagens (MMP-1 or interstitial collagenase, MMP-8 or neutrophil collagenase, MMP-13 or collagenase 3, or MMP-18 or collagenase 4), anti-gelatinases which decompose collagen of type IV or any form of denatured collagen (MMP-2 or gelatinase A (72 kDa), or MMP-9 or gelatinase B (92 kDa)), anti-stromelysins (MMP-3 or stromelysin 1, MMP-10 or stromelysin 2, or MMP-11 or stromelysin 3), agents which inhibit matrilysin (MMP-7), anti-metalloelastase agents (MMP-12), and agents which inhibit membrane metalloproteinases (MMP-14, MMP-15, MMP-16 and MMP-17).

C. Molecules that Inhibit Angiogenesis

The invention includes administering an agent which inhibits angiogenesis in the disc in combination with a disc stabilization device for the treatment of a degenerative disc disorder. Blood vessels are often a distinctive sign of the disc degenerative process, as monocytes have been implicated as a source associated with disc degradation. Inhibition of new blood vessels in a damaged disc may decrease the amount of inflammation and promote healing of the disc.

In one embodiment of the invention, the anti-angiogenesis agent is an agent which inhibits the action of VEGF. An anti-VEGF agent may be administered to inhibit angiogenesis of the disc in combination with a disc stabilization device. Vascular endothelial growth factor (VEGF) plays a key role of in the regulation of normal and abnormal angiogenesis (Ferrara et al. (1997) Endocr. Rev. 18:4). The finding that the loss of even a single VEGF allele results in embryonic lethality points to an irreplaceable role played by this factor in the development and differentiation of the vascular system (Ferrara et al.). Examples of anti-VEGF antibodies are described in US Appln. No. 2005/0112126. Another example of an anti-VEGF antibody is bevacizumab (Avastin®; Genentech, Inc., South San Francisco, Calif.), which is a humanized monoclonal antibody directed against VEGF-A.

Another class of angiogenesis inhibitors is the small-molecule tyrosine kinase inhibitors (TKIs), which act by inhibiting receptor signaling and associated downstream events. VEGFR TKIs include vatalanib (PTK787/ZK-222584; Novartis and Schering AG), SU11248, and ZD6474. Vatalanib is an orally available small-molecule TKI that is a potent inhibitor of VEGFR-1 and VEGFR-2 (Thomas et al. (2003) Semin Oncol. 30 (suppl 6):32). SU11248 inhibits VEGFR-2 and PDGF (Mendel et al. (2003) Clin Cancer Res 9:327-337), while ZD6474 inhibits VEGFR-2, VEGFR-3, and, to some extent, the epidermal growth factor receptor (EGFR) (Wedge et al. (2002) Cancer Res 62:4645-4655).

Vascular-targeting agents (VTAs) are another class of agents that target tumor vasculature and may be used in the invention to prevent angiogenesis within the intervertebral disc. VTAs are acute-acting agents that produce vascular shutdown within a few hours of administration. Unlike classic angiogenesis inhibitors that inhibit new vessel formation, VTAs selectively target endothelial cells in the existing vasculature of tumors (Iyer et al. (1998) Cancer Res 58:4510; Kanthou and Tozer (2002) Blood 99:2060; and Goto et al. (2002) Cancer Res 62:3711).

Other examples of anti-angiogenic agents which may be used in the invention include, but are not limited to, angiogenin inhibitors; beta-fibroblast growth factor inhibitors; GM-CSF inhibitors; interleukin 2 (IL-2) inhibitors; interleukin 6 (IL-6) inhibitors; interleukin 8 (IL-8) inhibitors; prostaglandin inhibitors TGF-beta inhibitors; TNF inhibitors; vascular endothelial growth; factor inhibitors such as PIC11, C225, SU6668, and anti-KDR monoclonal antibodies; vascular P factor inhibitors. Other examples of inhibitors of angioneogenesis include 2-methoxyestradiol; AG3340 (prinomastat); angiostatin; anti Integrin alpha vbeta3; batimastat; captopril; carboxyamido-triazole; CM100; combretastatin; contortrostatin; curcumin; diphenylureas; endostatin; flavone acetic acid; genistein; human tumor inhibitors; IL-12; irsogladine; kringle 5 of plasminogen; latent antithrombin; LM-609; marimastat; mitoxantrone; neovastat aetherna; nigella saliva; p53 gene therapy; pentosan polysulfate; PF-4; PI-88; prelatent antithrombin; PSK; recombinant platelet factor 4; retinoids; scatter factor; spironolactone; squalamine; suramin and suramin analogues; tamoxifen; taxol; tecogalan; tie2 pathway; thrombospondin 1 and 2; TIP-1; TNP-470 (AGM-1470); vinblastine; vitamin E; and vitaxin.

D. Molecules that Promote Production of Extracellular Matrix

The invention includes administering a growth factor which promotes production of new extracellular matrix production in combination with a disc stabilization device for the treatment of a degenerative disc disorder. Examples of growth factors which are associated with extracellular matrix production include members of the TGFβ superfamily. Growth factors of the TGFβ superfamily include, but are not limited to, bone morphogenic proteins (BMPs), such as BMP2 and BMP7/OP-1.

The members of the BMP family belong to the larger TGF-β superfamily of soluble secreted proteins, which act as powerful regulators of development and tissue repair in both vertebrates and invertebrates. The BMPs share sequence homologies in their carboxy-terminal domains with TGF-βs themselves. They are synthesized as large precursors and the mature protein, derived from the carboxy-terminal region, is released after proteolytic cleavage. These 30-38 kDa dimeric proteins are divided into subfamilies according to their amino acid sequence similarities. The BMP family members are multifunctional morphogens that control the development and apoptosis of a variety of cell types including osteoblasts, chondroblasts, neural cells, and epithelial cells, for example osteogenic protein-1 (OP-1, also called BMP-7) induces formation of new bone and cartilage. Examples of BMP-2 and BMP-7 nucleic acids and polypeptides are described in US 20050136042, incorporated by reference herein.

Combination treatments using the above-mentioned agents, e.g., combined use of a molecule that inhibits pro-inflammatory cytokines and a molecule which promotes new extra-cellular matrix, for treating a degenerative intervertebral disc disorder are also included in the scope of the invention.

Antibody Modification

Antibodies used in the invention, like those described above, e.g., anti-TNF, anti-MMP, anti-IL, and anti-VEGF antibodies, may be modified for improvements, such as improved administration and/or efficacy. In some embodiments, the antibody, or antigen binding fragment thereof, is chemically modified to provide a desired effect, such as extended half life. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

In one embodiment, anti-cytokine pegylated antibodies and antibody fragments may generally be used to treat degenerative disc disorders by administration in combination with a disc stabilization device as described herein.

Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, antibodies which inhibit pro-inflammatory cytokines, or fragments thereof, can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion of the invention can also be derivatized or linked to another functional molecule (e.g., another peptide or protein). Accordingly, the antibodies and antibody portions of the invention are intended to include derivatized and otherwise modified forms of the antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a polypeptide possessing a desired biological activity. Such polypeptides may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

V. Cell Therapy

The method of the invention may be combined with cell therapy or tissue engineering techniques which promote healing of a degenerative disc. Autologous cells may be amplified in vitro to be used for tissue engineering, i.e., biological substitutes to restore, maintain or improve tissue functions. Tissue engineering includes the use of compositions which relate to biological substitutes to restore, maintain or improve tissue functions. Matrices may be used to deliver cells to desired sites in the body, to define the potential space for engineered tissue, and to guide the process of tissue development.

In one embodiment, the method of the invention comprises administering to a subject an autologous (from the same individual) or allogeneic cell (from same species), or a regenerative growth factor which restores or improves disc tissue. Examples of autologous or allogeneic cells which may be used include, but are not limited to, a chondrocyte, a mesenchymal stem cell, and an adipocytic stem cell. Healthy cells may be introduced into the disc that can at least partially repair damage done to the disc during the degenerative process. In some embodiments, these cells are introduced into the nucleus pulposus and ultimately produce new extracellular matrix for the nucleus pulposus. In another embodiment, these cells are introduced into the annulus fibrosus and produce new extracellular matrix for the annulus fibrosus.

Cells for cell therapy may be obtained from a number of sources, including an intervertebral disc (for example, either nucleus pulposus cells or annulus fibrosus cells) and non-disc tissue (for example, mesenchymal stem cells or chondrocytes). Chondrocytes may be obtained, for example, from the degenerative disc itself or from another intact disc, or, alternatively, from a non-disc cartilage source, such as the ear.

Cells may also be taken from the patient's disc itself and cultured in an appropriate environment (possibly three-dimensional, such as an alginate ball, porous PLA/TGA structure, or collagen foam). Adult autologous stem cells from the patient's bone marrow can also be used, taken from the iliac crest, for example, and then placed in a culture environment similar to those previously mentioned.

Stem cells may also be used to treat degenerative disc disorders. In one embodiment mesenchymal stem cells (MSCs) are used in conjunction with the implants of the invention.

MSCs provide a special advantage for administration into a degenerating disc because they can more readily survive the relatively harsh environment present in the degenerating disc; they have a desirable level of plasticity; and MSCs also have the ability to proliferate and differentiate into the desired cells.

MSCs are obtained from bone marrow, preferably autologous bone marrow, as well as from adipose tissue. Other types of stem cells which can be used include adipocytic stem cells (from adipose tissue) and umbilical stem cells.

The implantable cells used for this process can be obtained by means of various procedures: they can be cells taken from the patient's intervertebral disc, adult stem cells from the patient's bone marrow, or embryonic stem cells. In addition, the treatment unit should include a means for injecting the cells into the disc, which can be a cannula type of syringe.

The invention may also include the device and methods described PCT/FR2003/003929 (WO 04/073532; FR 0300555; AU3303927). Such cell-based techniques which stimulate regeneration consist of implanting cells into the injured disc, which serve to regenerate the disc. The implanted cells may be of the same type as those composing the disc, or may be of another type. PCT/FR2003/003929 describes a unit for treating the degeneration of an injured intervertebral disc between two vertebrae, which includes cells which may or may not be from the same intervertebral disc and can be implantable in that disc and an intervertebral implant comprising an intervertebral wedge intended for placement between said vertebrae to limit the stresses applied to the disc; and a means of attachment for fastening the wedge to the vertebrae. The cell-based methods and compositions described in WO 04/073532 are incorporated by reference herein.

The invention may also include the device and methods described in FR0405611 (WO 05/118015) which describes a pedicle screw-based disc stabilization device which is used in combination with cells implanted in the intervertebral disc. The disc stabilization device permits regeneration of the invertebral disc's cells, wherein the presence of the stabilization device that is screwed into the vertebral maintains a space between them thus limiting the mechanical stresses applied to the intervertebral disc during treatment. The extradiscal stabilization implant may comprise cells which may or may not be from the same intervertebral disc and may be implanted in that disc and at least one device for stabilizing the two vertebrae. In one embodiment, the device includes two screws, (each screw intended to be screwed into one of the vertebrae), an elongated connecting piece, and two attachment devices for connecting the head of each screw to one of the ends of the connecting piece. In addition, since the stabilization device is screwed into the vertebrae, the invention can be used for patients who have undergone a total or partial resection of the vertebrae's spinous apophyses or whose spinous apophyses have very poor bone quality (due to osteoporosis, for example). It can also be used for the intervertebral disc between the fifth lumbar and the sacrum, since the first vertebra on the latter generally does not have a spinous apophysis. The invention can therefore be used in cases where interspinous process-based stabilization cannot be accomplished due to physiological reasons. Other types of intervertebral

VI. Administration

The therapeutic agent may be administered using a sustained release device, i.e., sustained delivery device. The sustained release device is adapted to remain within the disc for a prolonged period and slowly release the therapeutic agent contained therein to the surrounding environment. This mode of delivery allows the therapeutic agent to remain in therapeutically effective amounts within the disc for a prolonged period. The sustained release device provides controlled release of the therapeutic agent. In some embodiments, it provides continuous release. The sustained release device may provide intermittent release, and, further, may comprise a biosensor. Other release modes may also be used. In one embodiment, the therapeutic agent is predominantly released from the sustained delivery device by its diffusion through the sustained delivery device, e.g., through a polymer. In other embodiments, the therapeutic agent is predominantly released from the sustained delivery device by the biodegradation of the sustained delivery device (e.g., biodegradation of a polymer).

In other embodiments, the sustained release device comprises a bioresorbable material whose gradual erosion causes the gradual release of the therapeutic agent to the disc environment. The sustained release device may comprise a bioresorbable polymer, including, for example, a bioresorbable polymer which has a half-life of at least one month, more preferably at least two months, more preferably at least 6 months. In one embodiment, the sustained delivery device comprises bioerodable macrospheres. The therapeutic agent is preferably contained in a gelatin (or water or other solvent) within the capsule, and is released to the disc environment when the outer shell has been eroded. The device can include a plurality of capsules having outer shells of varying thickness, so that the sequential breakdown of the outer shells provides periodic release of the therapeutic agent.

The sustained delivery device may comprise a plurality of water-containing chambers, each chamber containing the therapeutic agent. A chamber is defined by bilayer lipid membranes comprising synthetic duplicates of naturally occurring lipids. Release of the drug can be controlled by varying at least one of the aqueous excipients, the lipid components, and the manufacturing parameters. Preferably, the formulation comprises no more than 10% lipid, such as, for example, Depofoam™ technology (Skyepharma PLC; London, United Kingdom).

In some embodiments, the sustained delivery device comprises the co-polymer poly-DL-lactide-co-glycolide (PLG). Preferably, the formulation is manufactured by combining the cycline compound, the co-polymer and a solvent to form a droplet, and then evaporating the solvent to form a microsphere. A plurality of microspheres are then combined in a biocompatible diluent. Preferably, the cycline compound is released from the co-polymer by its diffusion there through and by the biodegradation of the co-polymer (such as ProLease™ technology (Alkermes).

In one embodiment of the invention, hydrogel as a drug carrier to deliver the therapeutic agent. Hydrogel may be used as a drug carrier in addition to or instead of its use as an intradiscal implant, e.g., an artificial nucleus pulposus. A sustained release device may comprise a hydrogel, such that hydrogel is used to deliver the therapeutic agent in a time-release manner to the disc environment. Hydrogel employed in this invention may rapidly solidify to keep the desired agent at the application site, thereby eliminating undesired migration from the disc. The hydrogel used in the invention is also biocompatible, e.g., not toxic, to cells suspended in the hydrogel. Alternatively, an artificial nucleus pulposus may be made from a biomaterial such as, but not limited to, collagen type I, chytosan, fibrin, alginate, hyaluronate, cellulose, glycolide (PGA), polylactide (PLA) foam, and polyacrilonitril.

The additional therapeutic agent of the invention may be administered locally by direct injection into the subject with the damaged disc. In relation to the implantation of the stabilization disc device, the disc implant may be implanted in the subject prior to, concurrent with, or following administration of the therapeutic agent. The agents described in the invention may be combined with a drug carrier or drug delivery system which is optimized for treatment of a degenerative spinal disorder.

Therapeutic agents, as well as an artificial nucleus pulposus, may be administered directly to the disc. For example, a blunt tipped needle or cannula could be forced through the annulus. Upon withdraw of the needle, after injecting the agent into the nucleus pulposus and/or injecting an artificial nucleus pulposus, the separated fibers of the lamella would return to their normal position, sealing the annulus. The annulus fibrosis is thicker in the anterior and lateral portion of the disc. Thus, in a preferred embodiment, the needle would be inserted into the anterior or lateral portion of the disc. Those skilled in the art will realize the needle could be directed into the lateral portion of the disc percutaneously with fluourscopic guidance and into the anterior portion of the disc laparoscopically.

In addition to the above-mentioned delivery means, the antibodies and antibody-portions of the present invention can be also administered by a variety of methods known in the art, although for many therapeutic applications. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In addition, the agents may be administered using gene therapy techniques, wherein cells are transfected in vitro with DNA encoding the agent. Alternatively, viral or non-viral transfecting agents could be injected into the degenerative disc in order to modify the disc's chondrocytes. Nucleic acids encoding the molecules of the invention can be inserted into vectors and administered used as gene therapy vectors. Gene therapy vectors can be delivered to a subject by, for example, intravenous injection, local administration (see, e.g., U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Stem cells may be administered by injecting them into the disc in either an unconcentrated form or a concentrated form. When provided in concentrated form, they may be uncultured. Uncultured, concentrated stem cells can be readily obtained by centrifugation, filtration, or immunoabsorption.

VII. Degenerative Spinal Disorders

As used herein, the term “degenerative intervertebral disc disorder” refers to any condition relating to, i.e., caused by or resulting from, a degenerated disc. Degeneration of the intervertebral disc is believed to be a common cause of final pathology and of back pain.

As the intervertebral disc ages, it undergoes degeneration. The changes that occur are such that in many respects the composition of the nucleus seems to approach that of the inner annulus fibrosus. Intervertebral disc degeneration is, at least in part, the consequence of the composition change of the nucleus. It has been found that both the molecular weight and the content of proteoglycans from the nucleus decreases with age, especially in degenerated discs, and the ratio of keratin sulphate to chondroitin sulphate in the nucleus increases. These changes cause the nucleus to lose its water binding capability and its swelling pressure. As a result, the nucleus becomes less hydrated, and its water content drops from over 85% in preadolescence to about 70-75% in middle age. The glycosaminoglycan content of prolapsed discs has been found to be lower, and the collagen content higher than that of normal discs of a comparable age. Discs L-4-L-5 and L-5-S-1 are usually the most degenerated discs, resulting in lower back pain.

The spinal disc may be displaced or damaged due to trauma or a disease process. In this case and in the case of disc degeneration, the nucleus pulposus may herniate and/or protrude into the vertebral canal or intervertebral foramen, in which case it is known as a herniated or “slipped” disc. This disc may in turn press upon the spinal nerve that exits the vertebral canal through the partially obstructed foramen, causing pain or paralysis in the area of its distribution. The most frequent site of occurrence of a herniated disc is in the lower lumbar region. A disc herniation in this area often involves the inferior extremities by compressing the sciatic nerve.

Other disorders in which the methods and compositions of the invention may be used to treat include, but are not limited to, a herniated disc, including a voluminous herniated disc; a recurrent herniated disc; a herniated disc accompanying an L5 sacralization transitional anomaly; degenerative disc disease at a segment adjacent to fusion; modic I degenerative disease; and lumbar canal stenosis treated by partial laminectomy.

Degeneration of the spine can lead to other serious conditions as well. Lumbar spinal stenosis is a disease caused by a gradual narrowing of the spinal canal, which can put pressure on the nerve roots and spinal cord. A herniated disc, also known as a “slipped,” “ruptured” or “torn” disc, describes an abnormality of the intervertebral disc. Aging causes intervertebral discs to lose their flexibility, elasticity and shock-absorbing characteristics. Degenerative diseases can cause several different symptoms, including back pain, leg pain, weakness and numbness.

In addition, an IDP device, in accordance with the compositions and methods of the invention, may be used to relieve symptoms of lumbar spinal stenosis by distracting the affected spinal segment and maintaining the spine in a slightly flexed position, thus preventing extension or narrowing of the spinal canal. An IPD device may be used to relieve pressure and restrictions on the blood vessels and nerves caused by lumbar stenosis.

The method of the invention is used to treat disorders associated with degenerative spinal disorders, in addition to the degenerative disorder itself.

### EQUIVALENTS

Although the foregoing invention has been described in detail for purposes of clarity of understanding, it will be obvious that certain modifications may be practiced within the scope of the appended claims.

All publications and patent documents cited herein, as well as text appearing in the sequence listing, are hereby incorporated by reference in their entirety for all purposes to the same extent as if each were so individually denoted.

What is claimed is:

1. A method of treating a degenerative intervertebral disc disorder comprising implanting a disc stabilization device into a subject suffering from the disorder and administering at least one therapeutic agent which promotes healing of the disc to the subject such that treatment of the disorder occurs, wherein the agent is selected from the group consisting of
a) an agent which inhibits pro-inflammatory cytokines;
b) an anti-enzymatic agent which inhibits degradation of the extracellular matrix of the disc;
c) an agent which inhibits angiogenesis in the disc; and
d) a growth factor which promotes extracellular matrix production.

2. A method of treating a degenerative spinal disorder comprising administering a therapeutic agent which promotes healing of the disc to a subject who has a disc stabilization device, wherein the agent is selected from the group consisting of
a) an agent which inhibits pro-inflammatory cytokines;
b) an anti-enzymatic agent which inhibits degradation of the extracellular matrix of the disc;
c) an agent which inhibits angiogenesis in the disc; and
d) a growth factor which promotes extracellular matrix production.

3. The method of claims 1 or 2, wherein the disc stabilization device is load bearing.

4. The method of any one of claims 1-3, wherein disc stabilization device is an extradiscal stabilization device or an intradiscal stabilization device.

5. The method of claim 4, wherein the extradiscal device is an interspinous process-based device or a pedicle screw-based device.

6. The method of claim 5, wherein the interspinous process-based device is selected from the group consisting of an interspinous spacer, an interspinous process decompression (IPD) device, and a U-shaped interspinal device.

7. The method of claim 6, wherein the interspinous spacer comprises an elastically, deformable wedge which is inserted between two spinous processes and has two lateral walls and two opposite grooves in which the spinous processes engage.

8. The method of claim 7, wherein the interspinous spacer further comprises a fixing tie.

9. The method of claim 6, wherein the interspinous wedge further comprises
a) a fixing tie for retaining the spinous processes in the grooves;
b) a removable self-locking fixing member having first connecting means and through which the tie can slide when it moves in translation in a first direction, the self-locking fixing member being adapted to immobilize the tie against movement in translation in a second direction opposite to the first direction; and
c) at least one of the lateral wall so the first direction causing the spinous process to be clamped in the groove and the tie to be immobilized against movement in; and
d) at least one of the lateral walls of the wedge includes a second connecting means to cooperate with the first connecting means to connect the removable self-locking fixing member to the lateral wall, a movement of the free end of the tie to move the tie in translation in the first direction causing the spinous processes to be clamped in the groove and the tie to be immobilized against movement in translation relative to the block in the second direction.

10. The method of claim 5, wherein the pedicle screw-based device is selected from the group consisting of a dynamic external spacer stabilization system, a flexible jointed rod, a helical rod, a device for intervertebral assisted motion, and a total posterior spine system.

11. The method of any one of claims 5-10, wherein the extradiscal implant is coupled with a biocompatible hydrogel.

12. The method of claim 11, wherein the hydrogel comprises the therapeutic agent.

13. The method of any one of claims 1-3, wherein disc implant is an intradiscal stabilization implant.

14. The method of claim 13, wherein the intradiscal implant comprises hydrogel.

15. The method of claim 10, wherein the intradiscal implant is an artificial nucleus pulposus which supports or replaces the existing nucleus pulposus, or a portion thereof, of the intervertebral disc.

16. The method of claim 15, wherein the artificial nucleus pulposus contains a load bearing polymer.

17. The method of claim 16, wherein the artificial nucleus pulposus contains a non-load bearing polymer.

18. The method of any one of claims 15-17, wherein the artificial nucleus pulposus comprises a biocompatible hydrogel.

19. The method of any one of claims 15-17, wherein the artificial nucleus pulposus comprises a biomaterial selected from the group consisting of collagen type I, chytosan, fibrin, alginate, hyaluronate, cellulose, glycolide (PGA), polylactide (PLA) foam, and polyacrilonitril.

20. The method of any one of claims 1-19, wherein the agent which inhibits pro-inflammatory cytokines comprises a TNFα inhibitor or an anti-IL1 inhibitor.

21. The method of claim 20, wherein the anti-TNFα inhibitor is an antibody, or antigen binding portion thereof.

22. The method of claim 21, wherein the anti-TNFα antibody is a an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

23. The method of claim 21, wherein the anti-TNFα antibody is an isolated human antibody, or an antigen-binding portion thereof, with the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

24. The method of claim 21, wherein the anti-TNFα antibody is an isolated human antibody, or an antigen-binding portion thereof, with a light chain variable region (LCVR) comprising the amino acid sequence of SEQ ID NO: 1 and a heavy chain variable region (HCVR) comprising the amino acid sequence of SEQ ID NO: 2.

25. The method of claim 21, wherein the anti-TNFα antibody, or antigen-binding portion thereof, is Humira (D2E7; adalimumab), golimumab, or Remicade (infliximab).

26. The method of claim 20, wherein the anti-TNFα inhibitor is a fusion protein comprising Enbrel (etanercept).

27. The method of any one of claims 20, wherein the anti-IL1 inhibitor is an antibody, or antigen binding portion thereof.

28. The method of any one of claims 1-19, wherein the growth factor is a member of the TGFβ superfamily.

29. The method of claim 28, wherein the growth factor is BMP2 or BMP7.

30. The method of any one of claims 1-19, wherein the agent which inhibits angiogenesis inhibits VEGF.

31. The method of any one of claims 1-19, wherein the anti-enzymatic agent is an anti-aggrecanase or an anti-metalloproteinase.

32. The method of claim 31, wherein the anti-aggrecanase agent is directed to ADAMTS5.

33. The method of any one of claims 1-32, wherein the therapeutic agent is delivered using a delivery means selected from the group consisting of direct injection, implantation with a drug delivery implant, and gene therapy.

34. The method of any one of claims 1-33, further comprising administering an autologous cell or a regenerative growth factor which restores or improves disc tissue.

35. The method of claim 34, wherein the cell is selected from the group consisting of a chondrocyte, a mesenchymal stem cell, and an adipocytic stem cell.

36. The method of claim 35, wherein the chondrocytes are obtained from at least one source selected from the group consisting of the degenerative disc, an intact non-degenerative disc, and a non-disc cartilaginous source.

37. The method of any one of claims 1-36, wherein the disorder is degenerative disc disease.

38. A method of improving the disc quality of a damaged or degenerated intervertebral disc in a subject suffering from a degenerative intervertebral disc disorder comprising
a) decreasing the load of the disc through a load bearing disc stabilization device in the subject; and
b) administering a therapeutic agent to the subject, wherein then agent is selected from the group consisting an agent which inhibits pro-inflammatory cytokines;
an anti-enzymatic agent which inhibits degradation of the extracellular matrix of the disc; an agent which inhibits angiogenesis in the disc; and a growth factor which promotes extracellular matrix production.

39. A method of promoting a healing environment for the treatment of a damaged or degenerative disc in a subject comprising
a) decreasing the load of the disc through a load bearing disc stabilization device in the subject; and
b) administering a therapeutic agent to the subject, wherein then agent is selected from the group consisting an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production.

40. The method of claim 38 or 39, wherein the disc stabilization device is implanted in the subject prior to, concurrent with, or following administration of the therapeutic agent.

41. The method of claim 38 or 39, wherein the disc stabilization device is an extradiscal stabilization device or an intradiscal stabilization device.

42. The method of claim 41, wherein the extradiscal device is an interspinous process-based device or a pedicle screw-based device.

43. The method of claim 42, wherein the interspinous process-based device is selected from the group consisting of an interspinous spacer, an interspinous process decompression (IPD) device, a device for intervertebral assisted motion, and a U-shaped interspinal device.

44. The method of claim 39, wherein the intervertebral device comprises an elastically, deformable wedge which is inserted between two spinous processes and has two lateral walls and two opposite grooves in which the spinous processes engage.

45. The method of claim 44, wherein the interspinous spacer further comprises a fixing tie.

46. The method of claim 43, wherein the interspinous spacer further comprises
a) a fixing tie for retaining the spinous processes in the grooves;
b) a removable self-locking fixing member having first connecting means and through which the tie can slide when it moves in translation in a first direction, the self-locking fixing member being adapted to immobilize the tie against movement in translation in a second direction opposite to the first direction; and
c) at least one of the lateral wall so the first direction causing the spinous process to be clamped in the groove and the tie to be immobilized against movement in; and
d) at least one of the lateral walls of the wedge includes a second connecting means to cooperate with the first connecting means to connect the removable self-locking fixing member to the lateral wall, a movement of the free end of the tie to move the tie in translation in the first direction causing the spinous processes to be clamped in the groove and the tie to be immobilized against movement in translation relative to the block in the second direction.

47. The method of claim 42, wherein the pedicle screw-based device is selected from the group consisting of a dynamic external spacer stabilization system, a flexible jointed rod, a helical rod, a device for intervertebral assisted motion, and a total posterior spine system.

48. The method of claim 41, wherein the intradiscal device comprises a biocompatible hydrogel.

49. The method of claim 41, wherein the intradiscal device is an artificial nucleus pulposus which supports or replaces the existing nucleus pulposus, or a portion thereof, of the intervertebral disc.

50. The method of claim 49, wherein the artificial nucleus pulposus comprises a biocompatible hydrogel.

51. The method of claim 50, wherein the artificial nucleus pulposus comprises a biomaterial selected from the group consisting of collagen type I, chytosan, fibrin, alginate, hyaluronate, cellulose, glycolide (PGA), polylactide (PLA) foam, and polyacrilonitril.

52. The method of any one of claims 38-51, wherein the agent which inhibits pro-inflammatory cytokines comprises a TNFα inhibitor or an anti-IL1 inhibitor.

53. The method of claim 52, wherein the anti-TNFα inhibitor is an antibody, or antigen binding portion thereof.

54. The method of claim 53, wherein the anti-TNFα antibody is a an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

55. The method of claim 53, wherein the anti-TNFα antibody is an isolated human antibody, or an antigen-binding portion thereof, with the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

56. The method of claim 53, wherein the anti-TNFα antibody is an isolated human antibody, or an antigen-binding portion thereof, with a light chain variable region (LCVR) comprising the amino acid sequence of SEQ ID NO: 1 and a heavy chain variable region (HCVR) comprising the amino acid sequence of SEQ ID NO: 2.

57. The method of claim 53, wherein the anti-TNFα antibody is Humira (D2E7;
adalimumab) or Remicade (infliximab).

58. A method of treating degenerative disc disease comprising implanting an intradiscal stabilization device into a damaged or degenerated intervertebral disc of a subject suffering from a degenerative spinal disorder and administering a therapeutic agent which inhibits the inflammatory process associated with the damaged or degenerated disc.

59. The method of claim 58, wherein the intradiscal device comprises a biocompatible hydrogel.

60. The method of claim 58 or 59, wherein the intradiscal device is implanted into the subject prior to, concurrent with, or following the inhibition of the inflammatory process.

61. The method of any one of claims 58-60, wherein the intradiscal device is an artificial nucleus pulposus which supports or replaces the existing nucleus pulposus, or a portion thereof, of the intervertebral disc.

62. The method of claim 61, wherein the artificial nucleus pulposus comprises a biocompatible hydrogel.

63. The method of claim 62, wherein the artificial nucleus pulposus comprises a biomaterial selected from the group consisting of collagen type I, chytosan, fibrin, alginate, hyaluronate, cellulose, glycolide (PGA), polylactide (PLA) foam, and polyacrilonitril.

64. The method of any one of claims 58-63, further comprising implanting an extradiscal stabilization device.

65. The method of claim 59-62, wherein the hydrogel comprises an agent which inhibits pro-inflammatory cytokines;

66. A method of treating degenerative disc disease comprising implanting an extradiscal stabilization device into a damaged or degenerated intervertebral disc of a subject suffering from a degenerative spinal disorder and administering a therapeutic agent which inhibits the inflammatory process associated with the damaged or degenerated disc.

67. The method of claim 66, wherein the extradiscal device is an interspinous process-based device or a pedicle screw-based device.

68. The method of claim 67, wherein the interspinous process-based device is selected from the group consisting of an interspinous spacer, an interspinous process decompression (IPD) device, a device for intervertebral assisted motion, and a U-shaped interspinal device.

69. The method of claim 62, wherein the intervertebral implant comprises an elastically, deformable wedge which is inserted between two spinous processes and has two lateral walls and two opposite grooves in which the spinous processes engage.

70. The method of claim 69, wherein the interspinous spacer further comprises a fixing tie.

71. The method of claim 68, wherein the interspinous spacer comprises
a) a fixing tie for retaining the spinous processes in the grooves;
b) a removable self-locking fixing member having first connecting means and through which the tie can slide when it moves in translation in a first direction, the self-locking fixing member being adapted to immobilize the tie against movement in translation in a second direction opposite to the first direction; and
c) at least one of the lateral wall so the first direction causing the spinous process to be clamped in the groove and the tie to be immobilized against movement in; and
d) at least one of the lateral walls of the wedge includes a second connecting means to cooperate with the first connecting means to connect the removable self-locking fixing member to the lateral wall, a movement of the free end of the tie to move the tie in translation in the first direction causing the spinous processes to be clamped in the groove and the tie to be immobilized against movement in translation relative to the block in the second direction.

72. The method of any one of claims 66-71, wherein the extradiscal device is coupled with a biocompatible hydrogel.

73. The method of claim 72, wherein the hydrogel comprises the therapeutic agent.

74. The method of any one of claims 58-73, wherein the therapeutic agent inhibits pro-inflammatory cytokines.

75. The method of any one of claims 58-74, wherein the therapeutic agent is administered using a delivery means selected from the group consisting of direct injection, implantation with a drug delivery implant, and gene therapy.

76. The method of claim 75, further comprising administering an additional therapeutic agent selected from the group consisting of an anti-enzymatic agent which inhibits degradation of the extracellular matrix of the disc; an agent which inhibits angiogenesis in the disc; and a growth factor which promotes extracellular matrix production.

77. An intradiscal device for promoting healing of a damaged or degenerated intervertebral disc comprising a biocompatible hydrogel and a therapeutic agent which promotes healing of the disc, wherein the therapeutic agent is selected from the group consisting of an agent which inhibits pro-inflammatory cytokines; an anti-enzymatic agent which inhibits degradation of the extracellular matrix of the disc; an agent which inhibits angiogenesis in the disc; and a growth factor which promotes extracellular matrix production.

78. The intradiscal device of claim 77, wherein the hydrogel is load bearing.

79. The intradiscal device of claim 70, wherein the hydrogel is non-load bearing polymer.

80. The intradiscal device of any one of claims 70-72, wherein the agent which inhibits pro-inflammatory cytokines comprises a TNFα inhibitor or an anti-IL1 inhibitor.

81. The intradiscal device of claim 73, wherein the anti-TNFα inhibitor is an antibody, or antigen binding portion thereof.

82. The intradiscal device of claim 74, wherein the anti-TNFα antibody is a an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

83. The intradiscal device of claim 74, wherein the anti-TNFα antibody is an isolated human antibody, or an antigen-binding portion thereof, with the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

84. The intradiscal device of claim 74, wherein the anti-TNFα antibody is an isolated human antibody, or an antigen-binding portion thereof, with a light chain variable region (LCVR) comprising the amino acid sequence of SEQ ID NO: 1 and a heavy chain variable region (HCVR) comprising the amino acid sequence of SEQ ID NO: 2.

85. The intradiscal device of claim 74, wherein the anti-TNFα antibody is Humira (D2E7; adalimumab) or Remicade (infliximab).

86. The intradiscal device of claim 73, wherein the anti-TNFα inhibitor is Enbrel (etanercept).

87. The intradiscal device of claim 73, wherein the anti-IL1 agent is an antibody, or antigen binding portion thereof.

88. The intradiscal implant of any one of claims 77-79, wherein the growth factor is a member of the TGFβ superfamily.

89. The intradiscal implant of claim 88, wherein the growth factor is BMP2 or BMP7.

90. The intradiscal implant of any one of claims 77-79, wherein the agent which inhibits angiogenesis inhibits VEGF.

91. The intradiscal implant of any one of claims 77-79, wherein the anti-enzymatic agent is an anti-aggrecanase or an anti-metalloproteinase.

92. The intradiscal implant of claim 91, wherein the anti-aggrecanase agent is directed to ADAMTS5.

93. The intradiscal implant of any one of claims 77-79, wherein the therapeutic agent is delivered using a delivery means selected from the group consisting of direct injection, implantation with a drug delivery implant, and gene therapy.

94. The intradiscal implant of any one of claims 77-79, wherein the implant comprises an artificial nucleus pulposus.

95. The intradiscal implant of any one of claims 77-79, which is designed to be injected in the disc.


SEQUENCE LISTING (37 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  D2E7 light chain variable region CDR3
  VARIANT 9: Xaa = Any Amino Acid
QRYNRAPYX

SEQ ID NO: 4 - protein, 12 aa, Artificial Sequence
  D2E7 heavy chain variable region CDR3
  VARIANT 12: Xaa = Any Amino Acid
VSYLSTASSLDX

SEQ ID NO: 5 - protein, 7 aa, Artificial Sequence
  D2E7 light chain variable region CDR2
AASTLQS

SEQ ID NO: 6 - protein, 17 aa, Artificial Sequence
  D2E7 heavy chain variable region CDR2
AITWNSGHIDYADSVEG

SEQ ID NO: 7 - protein, 11 aa, Artificial Sequence
  D2E7 light chain variable region CDR1
RASQGIRNYLA

SEQ ID NO: 8 - protein, 5 aa, Artificial Sequence
  D2E7 heavy chain variable region CDR1
DYAMH

SEQ ID NO: 9 - protein, 107 aa, Artificial Sequence
  2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  2SD4 light chain variable region CDR3
QKYNSAPYA

SEQ ID NO: 12 - protein, 9 aa, Artificial Sequence
  EP B12  light chain variable region CDR3
QKYNRAPYA

SEQ ID NO: 13 - protein, 9 aa, Artificial Sequence
  VL10E4 light chain variable region CDR3
QKYQRAPYT

SEQ ID NO: 14 - protein, 9 aa, Artificial Sequence
  VL100A9 light chain variable region CDR3
QKYSSAPYT

SEQ ID NO: 15 - protein, 9 aa, Artificial Sequence
  VLL100D2 light chain variable region CDR3
QKYNSAPYT

SEQ ID NO: 16 - protein, 9 aa, Artificial Sequence
  VLL0F4 light chain variable region CDR3
QKYNRAPYT

SEQ ID NO: 17 - protein, 9 aa, Artificial Sequence
  LOE5 light chain variable region CDR3
QKYNSAPYY

SEQ ID NO: 18 - protein, 9 aa, Artificial Sequence
  VLLOG7 light chain variable region CDR3
QKYNSAPYN

SEQ ID NO: 19 - protein, 9 aa, Artificial Sequence
  VLLOG9 light chain variable region CDR3
QKYTSAPYT

SEQ ID NO: 20 - protein, 9 aa, Artificial Sequence
  VLLOH1 light chain variable region CDR3
QKYNRAPYN

SEQ ID NO: 21 - protein, 9 aa, Artificial Sequence
  VLLOH10 light chain variable region CDR3
QKYNSAAYS

SEQ ID NO: 22 - protein, 9 aa, Artificial Sequence
  VL1B7 light chain variable region CDR3
QQYNSAPDT

SEQ ID NO: 23 - protein, 9 aa, Artificial Sequence
  VL1C1 light chain variable region CDR3
QKYNSDPYT

SEQ ID NO: 24 - protein, 9 aa, Artificial Sequence
  VL0.1F4 light chain variable region CDR3
QKYISAPYT

SEQ ID NO: 25 - protein, 9 aa, Artificial Sequence
  VL0.1H8 light chain variable region CDR3
QKYNRPPYT

SEQ ID NO: 26 - protein, 9 aa, Artificial Sequence
  LOE7.A light chain variable region CDR3
QRYNRAPYA

SEQ ID NO: 27 - protein, 12 aa, Artificial Sequence
  2SD4 heavy chain variable region CDR3
ASYLSTSSSLDN

SEQ ID NO: 28 - protein, 12 aa, Artificial Sequence
  VH1B11 heavy chain variable region CDR3
ASYLSTSSSLDK

SEQ ID NO: 29 - protein, 12 aa, Artificial Sequence
  VH1D8 heavy chain variable region CDR3
ASYLSTSSSLDY

SEQ ID NO: 30 - protein, 12 aa, Artificial Sequence
  VH1A11 heavy chain variable region CDR3
ASYLSTSSSLDD

SEQ ID NO: 31 - protein, 12 aa, Artificial Sequence
  VH1B12 heavy chain variable region CDR3
ASYLSTSFSLDY

SEQ ID NO: 32 - protein, 12 aa, Artificial Sequence
  VH1E4 heavy chain variable region CDR3
ASYLSTSSSLHY

SEQ ID NO: 33 - protein, 12 aa, Artificial Sequence
  VH1F6 heavy chain variable region CDR3
ASFLSTSSSLEY

SEQ ID NO: 34 - protein, 12 aa, Artificial Sequence
  3C-H2 heavy chain variable region CDR3
ASYLSTASSLEY

SEQ ID NO: 35 - protein, 12 aa, Artificial Sequence
  VH1-D2.N heavy chain variable region CDR3
VSYLSTASSLDN

SEQ ID NO: 36 - DNA, 321 bp, Artificial Sequence
  D2E7 light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  D2E7 heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt